(19) United States
(12) Patent Application Publication
(10) Pub. No.: US 2026/0060585 A1
(43) Pub. Date: Mar. 5, 2026

(54) ADJUSTABLE SENSOR LAYOUT INTERFACE FOR MAGNETIC FIELD MEASUREMENTS

(71) Applicant: Father Flanagan's Boys' Home Doing Business as Boys Town National Research Hospital, Omaha, NE (US)

(72) Inventors: Alexa Marie Wildy, Omaha, NE (US); Cooper Lee Livermore, Omaha, NE (US)

(21) Appl. No.: 19/318,981

(22) Filed: Sep. 4, 2025

Related U.S. Application Data
(60) Provisional application No. 63/690,357, filed on Sep. 4, 2024.

(51) Int. Cl. A61B 5/245; A61B 5/00; G01R 33/26

(52) U.S. Cl. CPC A61B 5/245; A61B 5/6803; G01R 33/26; A61B 2562/0223; A61B 2562/221

(57) ABSTRACT

Neural imaging helmets include the placement of a number of sensors, such as in a sensor array on and around the helmet. The placement and orientation of the sensors puts them close to the participant's head for best neural image capturing. As people's head size and shape varies, moving the sensors from one helmet to another can be onerous. Therefore, connecting the sensors and associated cables together allows for efficient removal and replacement of the sensors and cables. The use of sensor holders connected to another will aid in ensuring proper orientation and location, regardless of the helmet used. This will allow different sized helmets to be readily prepared for use with neural imaging devices.

### CROSS REFERENCE TO RELATED APPLICATIONS

This application claims priority under 35 U.S.C. § 119(e) to provisional patent application U.S. Ser. No. 63/690,357, filed Sep. 4, 2024. The provisional patent application is hereby incorporated by reference in its entirety herein, including without limitation: the specification, claims, and abstract, as well as any figures, tables, appendices, or drawings thereof.

### TECHNICAL FIELD

The present disclosure relates generally to the field of neuroscience. More particularly, but not exclusively, the disclosure discloses systems, methods, and apparatus including a sensor array for use with head covering devices used with neuroimaging and neurostimulation devices, such as optically pumped magnetometers.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

OPM-MEG (Optically Pumped Magnetometers-Magnetoencephalography (MEG) is a new non-invasive technology for imaging brain function in real-time with several advantages over traditional MEG. The OPM includes a number of sensors, which may be over 100 sensors, which are used with a participant. These OPM sensors serve the same role as the magnetometers in the traditional cryogenic magnetoencephalography (MEG) scanner, although with different mechanisms of action. Both the MEG and OPM are used to sense the small magnetic field generated by brain activity. The MEG scanning system has become a widely used tool in neuroscience research, but due to it being a large unmovable machine, participants are required to be as still as possible making certain populations, especially children, difficult to obtain data from.

The OPM solves this issue by having the sensors placed inside a helmet worn by participants, allowing the participant with the helmet to move while maintaining the helmet's position with respect to the participants' head.

To ensure the best data, helmet size and shape is extremely important. It is ideal to have a helmet size and shape that corresponds to the individual participant's head size and shape because you want the sensors as close to the head as possible because of the inverse square law. This has been addressed by having a helmet library (i.e., a large number of helmets of varying size and shape), allowing each participant to have a well-fitting helmet. However, this has created a new problem, namely, the difficulty in changing the sensors from one helmet to another. The current helmet design requires three trained technicians working an hour to remove the sensors and place them into the new helmet in the proper location. To put sensors into a different helmet, the general steps are to remove the sensors from the holders on a first helmet, pull the cables out of the channels that they are in, undo all of the hook and loops that are holding the cables together, individually place the sensors in the second helmet, verifying the orientation with each sensor, reconnecting the cables together, and then putting the cables in the associated channel. Throughout this process it is common that a sensor accidentally gets put in backwards and cables may get slightly tugged out of their place, causing the sensors to no longer function or provide insufficient data acquisition. After switching helmets, the sensors must then be checked to ensure correct orientation and operation of the sensors, requiring an additional ˜1.5-5 hours.

Therefore, there is a need in the art for a helmet to allow a faster and more accurate switching process and design for the sensors of the neuroimaging device.

### SUMMARY

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of any of the aspects and/or embodiments of the present disclosure to improve the efficiency of moving an array of sensors from one head covering to another. For example, the improved efficiency will include both the time required and the accuracy of the movement of the sensor array.

It is still yet a further object, feature, and/or advantage of any of the aspects and/or embodiments of the present disclosure to manage both sensors and attached cables of the sensors for moving both the sensors and the cables from one head covering apparatus to another between uses with a neural imaging device.

It is still yet a further object, feature, and/or advantage of any of the aspects and/or embodiments of the present disclosure to ensure that sensors used with a head covering for use with a neural imaging device are placed as close to a participant's head as possible.

It is another object, feature, and/or advantage of any of the aspects and/or embodiments of the present disclosure to ensure appropriate orientation of sensors on a helmet used with a neural imaging machine.

The system and methods disclosed herein can be used in a variety of applications. For example, while the disclosure includes use with an OPM machine, it should be appreciated that any head covering that includes a number of sensors and/or a sensor array could benefit from the present disclosure.

It is preferred that the apparatus be safe, cost effective, and durable. For example, the disclosed sensor arrangement and/or array should be able to be moved from one head covering to another multiple times without needing to repair or replace the same.

At least one embodiment disclosed herein comprises a distinct aesthetic appearance. Ornamental aspects included in such an embodiment can help capture a consumer's attention and/or identify a source of origin of a product being sold. Said ornamental aspects will not impede functionality of the system.

Methods can be practiced which facilitate use, manufacture, assembly, maintenance, and repair of a sensor system and/or apparatus which accomplish some or all of the previously stated objectives.

The sensor system and/or apparatus can be incorporated into systems or kits which accomplish some or all of the previously stated objectives.

According to some aspects of the present disclosure, a helmet system for use with a neural imaging machine comprises a helmet base to cover at least a portion of a user's head; a plurality of sensor holders detachably connectable to the helmet base, each of the plurality of sensor holders configured to hold a sensor; and a substrate connected to the plurality of sensor holders to allow movement and selective attachment and detachment of the plurality of sensor holders as a substantially single unit.

According to at least some embodiments of the present disclosure, the helmet base comprises a plurality of pockets, wherein the pockets are sized and oriented to receive a portion of one of the plurality of sensor holders.

According to at least some embodiments of the present disclosure, the plurality of pockets each comprise an orientation key to aid in orientation of the sensor holders relative to the helmet base.

According to at least some embodiments of the present disclosure, each of the plurality of pockets include a rim that extends away from the helmet base.

According to at least some embodiments of the present disclosure, each of the plurality of sensor holders comprises one or more outwardly extending protrusions to hold the sensor therein.

According to at least some embodiments of the present disclosure, one or more of the outwardly extending protrusions comprises a retaining lip for holding the sensor in place.

According to at least some embodiments of the present disclosure, the plurality of sensor holders comprises a sensor key to orient the sensor relative to a sensor holder.

According to at least some embodiments of the present disclosure, each of the plurality of sensor holders comprises a cable management system to hold a portion of a cable connected to a corresponding sensor positioned in a sensor holder.

According to at least some embodiments of the present disclosure, the substrate comprises a flexible material.

According to at least some embodiments of the present disclosure, the substrate comprises a fabric.

According to additional aspects of the disclosure, a sensor system for use with a helmet of a neural imaging device comprises a plurality of sensor holders, each of the sensor holders comprising a base and one or more outwardly extending members, wherein each of the plurality of sensor holders are configured to hold a sensor such that the sensor is unable to substantially move relative to the sensor holder; and a flexible substrate connecting each of the plurality of sensor holders to one another.

According to at least some embodiments of the present disclosure, each base of the plurality of sensor holders comprises an asymmetrical shape.

According to at least some embodiments of the present disclosure, one or more of the outwardly extending protrusions of the plurality of sensor holders comprises a retaining lip for holding the sensor in place.

According to at least some embodiments of the present disclosure, each of the plurality of sensor holders comprises a cable management system to hold a portion of a cable connected to a corresponding sensor positioned in a sensor holder.

According to at least some embodiments of the present disclosure, the flexible substrate comprises a fabric material.

According to at least some embodiments of the present disclosure, the plurality of sensor holders and the flexible substrate are substantially non-ferromagnetic.

According to yet additional aspects of the disclosure, a neural imaging system comprises a helmet base comprising a plurality of pockets; a plurality of sensor holders detachably connectable to the helmet base at the plurality of pockets of the helmet base; a plurality of sensors positioned in the plurality of sensors, wherein the plurality of sensors associated with a neural imaging machine; and a substrate connected to the plurality of sensor holders.

According to at least some embodiments of the present disclosure, the plurality of pockets of the helmet base are associated with desired locations of the plurality of sensors of the neural imaging machine.

According to at least some embodiments of the present disclosure, each of the plurality of pockets and a base of each of the plurality of sensor holders comprise a corresponding asymmetrical shape to aid in orienting a sensor held in each of the plurality of sensor holders.

According to at least some embodiments of the present disclosure, each of the plurality of sensor holders comprises a cable management system to position a cable connected to a sensor of the plurality of sensors.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 is a perspective view of a helmet system for use with neural imaging machines according to aspects and/or embodiments of the present disclosure.

FIG. 2 is a perspective view of a helmet base of a helmet system according to aspects and/or embodiments of the present disclosure.

FIG. 3 is an enlarged view of a pocket on the helmet base.

FIG. 4 is a perspective view of a sensor holder including aspects of the present disclosure.

FIG. 5 is a perspective view of another sensor holder including aspects of the present disclosure.

FIG. 6 is a plan view of a base of a sensor holder.

FIG. 7 is a view of multiple sensor holder pockets of a helmet base.

FIG. 8 is a depiction of a neural imaging system including a helmet, sensor holders, and substrate, showing sensors and associated cables of the sensors positioned on the system according to aspects of the present disclosure.

FIG. 9 is a view showing sensor holders attached to a substrate with the sensor holders detached from a helmet base.

FIG. 10 is a view showing sensor holders attached to a substrate with the sensor holders attached to a helmet base.

An artisan of ordinary skill in the art need not view, within isolated figure(s), the near infinite distinct combinations of features described in the following detailed description to facilitate an understanding of the present disclosure.

### DETAILED DESCRIPTION

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

As will be understood, aspects and/or embodiments of the present disclosure relate to neuroimaging machines, such as an optically-pumped magnetometry-magnetoencephalogram (OPM-MEG) systems and the like. The term “neuroimaging system” or “machine” will be used to cover any type of neuroimaging system that is known and used to measure brain activity, including OPMs and magnetoencephalography (MEG) machines, which is another neuroimaging machine 10. In neuroimaging machines, a participant is engaged with the machine to allow the machine to acquire neural data, such as through a helmet or head covering. The neuroimaging machines utilize sensors to collect information from the participant.

As noted, OPMs utilize helmets or head coverings to place sensors close to the participant's head. Having helmets that are close in size and shape for the participant utilizing the helmet is also important so that the helmet does not move in position in relation to the head. If the helmet is too big, the position of the helmet on the head may change with any movement. The helmet's position on the head must be known to source localize, which is identifying which part of the brain the signal came from. If the helmet moves positions on the head during the scan the data would incorrectly identify where the signal was coming from in the brain.

Therefore, to address the issue of having to move sensors for a neuroimaging machine, such as an OPM, from one helmet size to another, the present disclosure includes systems and methods for timely and accurately being able to move the sensors. The disclosure also includes ways to ensure that the sensors are oriented correctly and to ensure that the cables associated with the sensors and machines are routed, which also improves the confidence in the sensors being moved from one helmet to another.

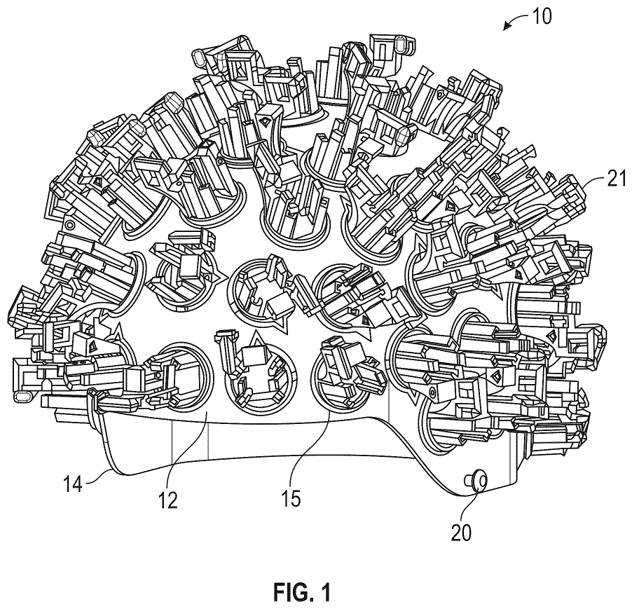

FIG. 1 is a view of a helmet system 10 for use with an OPM machine that includes aspects of embodiments of the present disclosure. In general, the helmet system 10 includes a helmet base 12 and a plurality of sensor holders 21 connected to the helmet base 12. The sensor holders 21 are used to house a plurality of sensors (one sensor per holder) that are used with the neuroimaging machine. The sensor holders 21 are removably or detachably connected to the helmet base 12 to allow quick and accurate removal and placement of the sensors via the holders from one helmet to another, such as changing the size of helmet based upon the participant's head size and/or shape. As will be understood, the use of the helmet base 12, sensor holders 21, as well as additional, optional aspects, will reduce the amount of time for moving the sensors from one helmet to another, while ensuring that the sensors are positioned accurately and safely for best data collection.

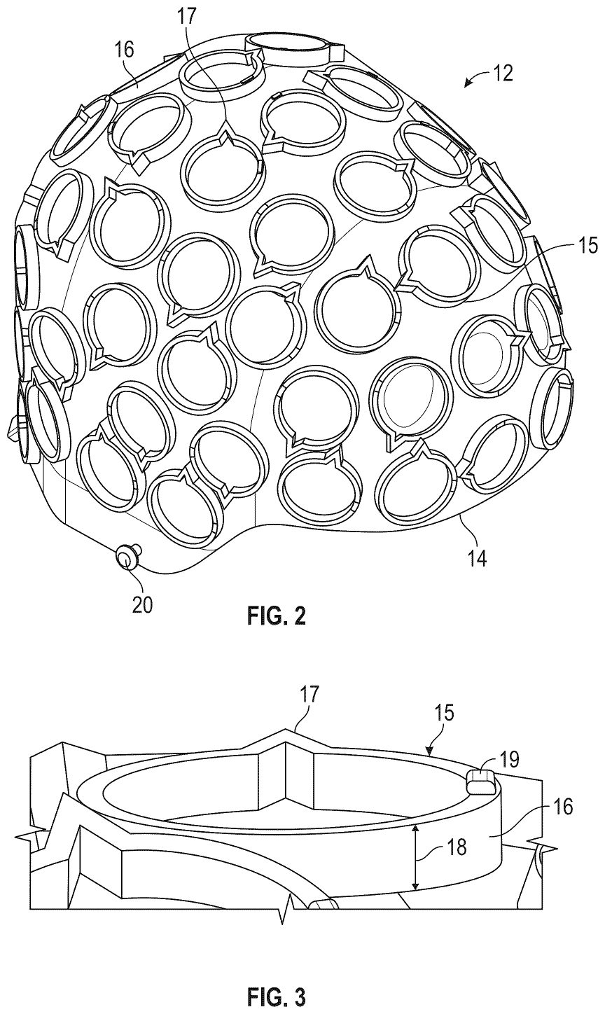

The helmet system 10 includes a helmet base 12, which is the portion of the system that is placed on the participant's head (not shown). The helmet base 12 includes a general shape of the helmet 14 and pockets 15 to hold sensor holders 21 onto the general helmet shape. FIG. 2 is a view of the helmet base 14 without any sensor holders 21, and better shows the layout of the pockets 15 on the helmet base 12. As will be understood, the pockets 15 are used in conjunction with sensor holders 21 to place, position, and orient the sensor holders 21 and sensors 52 relative to the helmet for use with the OPM or other neuroimaging machine.

The number of pockets 15 will generally coincide with the number of sensors used with a machine. However, good practice is to have as many pockets 15 in the helmet base 12 as there are number of sensor holders 21 and thus, sensors. According to at least some embodiments, there is ample space between adjacent pockets 15 for the sensor holders 21 to allow airflow around the sensors 52 for keeping the sensors cool and to allow space for a cable management system.

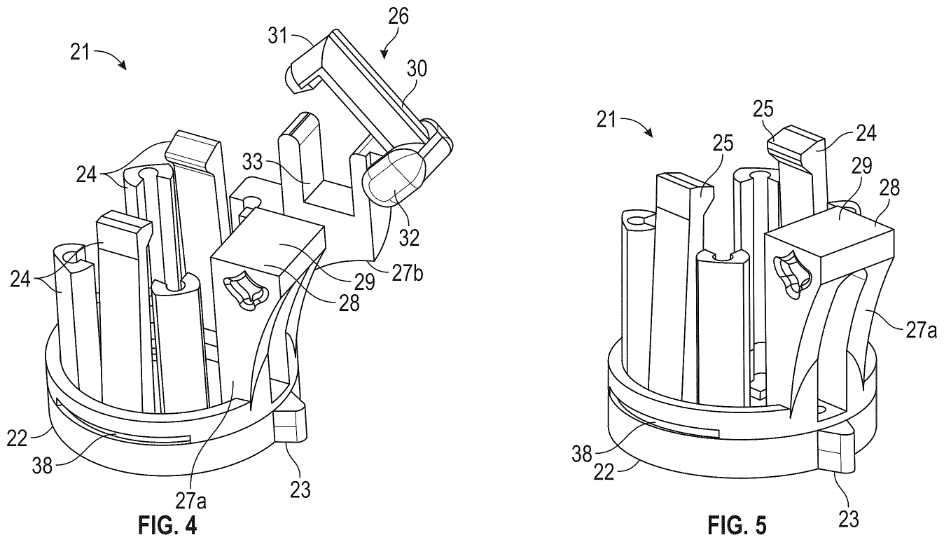

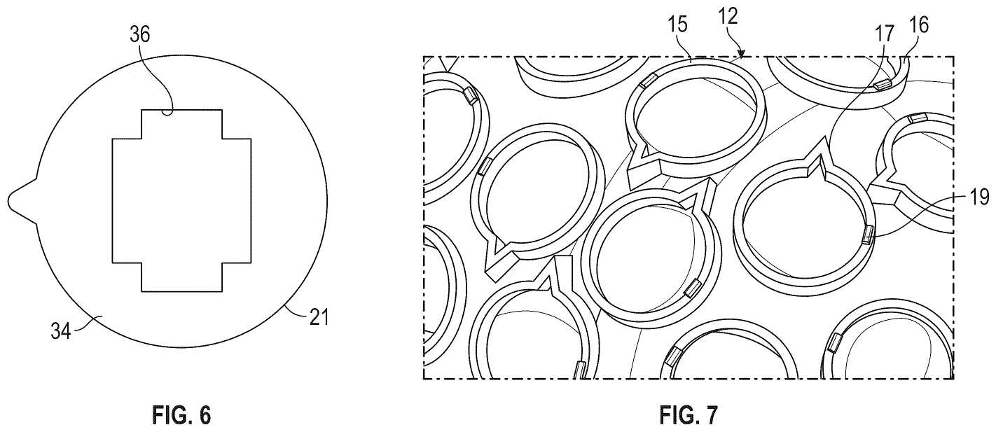

As shown in FIGS. 2 and 3, the pockets 15 are defined by a pocket rim 16, which includes a raised portion outlining the shape and size of the pocket 15. For example, the pocket rim 16 can be about the same size and shape as an outer perimeter of a bottom portion of a sensor holder 21 (see, e.g., FIGS. 4-6 showing the bottom portion or base 22 of the sensor holders 21). Note that the pocket shape includes an orientation key 17. As will be understood, the sensor holders 21 include a similar orientation member 23. The use of the keys 17, 23 will ensure that the sensor holders, and the sensors therein, are oriented correctly in relation to the helmet base 12 (see, e.g., FIGS. 6 and 7 showing corresponding shapes, including keys, of the sensor holders and the pockets).

The pocket 15 in the helmet base 12 is used to hold a sensor holder 21 such that the position of the sensor holder 21 does not change relative to the pocket 15, even when the helmet base 12 is rotated in any direction. The pocket 15 should be able to hold the sensor holder 21 such that the sensor holder 21 is not able to be pushed out the opposite side of the pocket 15 in which the sensor holder 21 entered the pocket 15 (i.e., the bottom of the pocket). The pocket 15 is a sort of protrusion away from the helmet base surface, and the distance of the protrusion can be the height 18 of the pocket 15.

According to at least some embodiments, the pocket rim 16 includes at least one lip or detent 19 at a distal edge of the rim 16 (see, e.g., FIG. 3). When a lip or detent 19 is used on the pocket rim, this will interact with a lip or ridge 38 of the sensor holder 21, such as at a base 22 or lower portion of the holder, which is shown in FIGS. 4 and 5. The interaction of the detent 19 and the ridge 38 will aid in holding the sensor holder in place relative to the pocket 15 of the helmet base 12.

The bottom of the pocket 15 may be solid or have holes. Having holes would allow for an air insulation pocket to reduce the amount of heat transfer from the sensors to the participant's head. Having holes may require the sensors be placed farther from the head to ensure the bottom of the pocket 15 will not break from repetitively putting the sensor holders in and out.

Referring back to the helmet base 12, it is preferred to have the shape of the base closely resemble the participant's head. It is preferred, but not required, to have the base of the helmet not deflect more than 3.5 cm when 18 N of force is distributed across a 20 cm\hat{ }2 area and applied to any part of the helmet while being fixed at any other part of the helmet. This is to verify that the helmet does not deflect when one puts the helmet on a head. Depending on the material used to make the base helmet, the thickness may need to change to not go over the maximum recommended deflection. With the 3D scanning technology and Motion Capture tracking equipment that exists today, it is recommended that the helmet does not deflect too much, but in the future when motion capture tracking, and/or 3D scanning equipment gets better, the rigidity of the helmet becomes less and less important.

The helmet base 12 may be 3D printed (or otherwise created using additive manufacturing), sculpted, carved, cut, molded, or formed out of any solid body(s) or body(s) that will harden into a solid body that is not magnetic. The material(s) used to make the helmet base 12 should be heat resistant up to at least the temperature that the surface(s) that touch the helmet base of the sensor holders 21 heat up to, but it is recommended to use a material that is heat resistant at least to the temperature that the OPMs get to.

Additionally, as shown in the figures, the helmet base may include at least one attachment point 20 for a chin strap (not shown) or another member. The chin strap or other member can be used to aid in fixing the helmet system 10 relative to a participant's head and to mitigate movement of the helmet relative to the head.

The sensor holders 21 are shown in FIGS. 4 and 5. As previously noted (and shown in FIG. 1), the number of sensor holders 21 will match the number of sensors 52 and the number of sensor holders 21 is limited by the surface area of the outer side of the helmet base 12. As will be understood, the sensor holder 21 holds the sensor 52 such that the position of the sensor in the holder does not change relative to the sensor holder 21, even when the sensor holder 21 is rotated or otherwise moved in any direction. This will ensure that the sensors are in a desired position and/or orientation.

The sensor holder 21 should be able to hold the sensor so that the sensor is not able to be pushed out the opposite side of the sensor holder 21 in which the sensor entered the sensor holder. One option to accomplish this is to have a full or partial thin base under the sensor in the sensor holder.

As shown in the figures, the sensor holder 21 includes a base 22. The base 22 includes a key 23. As noted, the key 23 is shaped relative to the pocket key 17 to aid in providing and ensuring a proper orientation for the sensor holder 21 relative to the helmet base 12. While a key 23 is shown, it should also be appreciated that the bottom or base 22 of the holder 21 be asymmetrical, with the pocket having a similar, asymmetrical shape, so that the base 22 of the holder 21 is only able to fit within the pocket in a desired orientation.

Extending away from the base 22 is one or more protrusions or arms 24. The protrusions 24 at least partially surround a sensor relative to the holder 21 and hold the sensor in place therein. The sensor 52 may be held in the sensor holder 21 with friction, balanced between at least two surface/protrusions, and/or mechanically locked in place.

In general, a mechanical fastener is a device that is used to mechanically join or fasten two or more objects together. In general, fasteners are used to create non-permanent joints or connections; that is, joints that can be removed or dismantled without damaging the joining components. General types of mechanical fasteners can include threaded (bolts, screws, nuts, studs, etc.) or non-threaded (keys, pins, retaining rings, etc.). Additional fasteners can include, but are not limited to nails, rivets, and the like. Non-mechanical fasteners may include adhesives, fittings, clearance fittings, friction fittings, compression fittings, transition fittings, snaps, snap fits, hook and loops, joints, and the like. For simplistic purposes, screws, nuts, bolts, pins, rivets, staples, washers, grommets, latches (including pawls), ratchets, clamps, clasps, flanges, ties, adhesives, welds, any other known fastening mechanisms, or any combination thereof may be used to facilitate fastening, may be used for any of the connections described herein and all are to be considered swappable with one another for any of the attachment, connection, and/or fastening of components, either temporarily or permanently. It is further considered that any combination of any of the listed mechanical and/or non-mechanical fasteners or methods of fastening are to be considered a part of the disclosure.

For examples, as shown in the figures, there are a number of protrusions 24, with at least two protrusions having a lip 25. The lips 25 are configured to extend inward (i.e., radially), and will cover a portion of the sensor in the holder to mitigate the sensor from moving away from the base 22 of the holder 21.

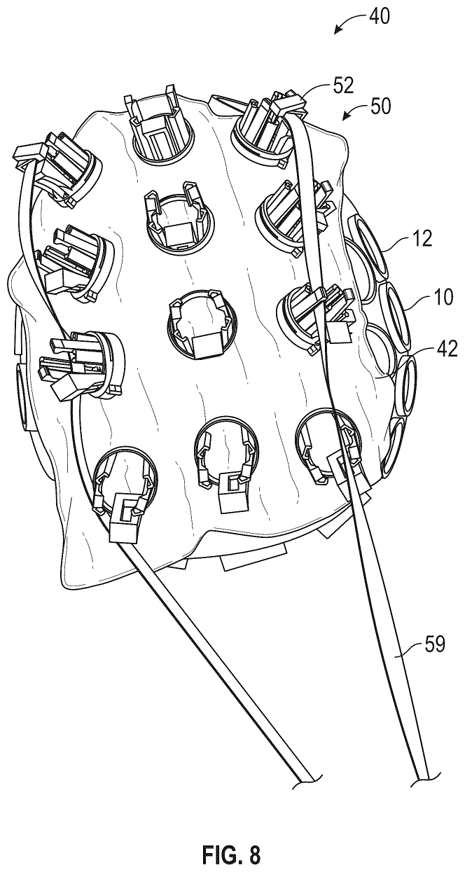

According to at least some embodiments, the sensor holders 21 include a cable management system 26. The cables 54 (see, e.g., FIG. 8) connect the sensors 52 to the neuroimaging machine. It is important to ensure that the cables 54 remain connected to the sensors 52 to make sure that the machine continues acquiring the data from the participant, even while moving. In addition, as part of moving the sensors 52 from one helmet to another, it is advantageous to maintain the connected cables to the sensors to reduce the amount of time required to move said sensors. Therefore, it is advantageous to have an attachment point for at least one cable on the sensor holder 21 itself. This is to mitigate the cable from getting tugged or bent close to the attachment point on the sensor 52. Having a cable management system 26 that holds multiple cables together either on the sensor holders 21 or on the helmet base 12 is also included in at least some embodiments but is not required in all such embodiments.

According to at least some embodiments, the cable management system 26 secures the cable 54 near where the cable comes out of a sensor 52 and secures said cable such that the cable does not bend near the sensor. Additional attachment points can be added to hold groups of cables together. According to at least some aspects and/or embodiments, the cable management system 26 can be either directly connected to the sensor holder 21 or indirectly connected to the sensor holder 21 via a flexible material/fabric connecting the sensor holders, as will be disclosed herein.

As shown in the FIGS. 4 and 5, examples of at least some cable management systems 26 are provided. The figures show a first cable member 27a and a second cable member 27b. The cable members are protrusions extending away from the base of a sensor holder 21. The first cable member 27a is a stand-like member that has a substantially flat top 28. A non-mechanical fastener, such as hook and loops 29, can be positioned on the substantially flat top 28 and a corresponding hook or loops member can be on the cable to temporarily attach the cable to the stand. The second cable member 27b is a clip-like member 30. The second cable member 27b comprises clip 30 including an arm 31 that is pivotally connected at a pivot 32 to the member 27b. When the arm 31 is closed, a channel 33 is formed within the cable member 27b. The channel 33 is able to hold a group of cables together to control the positioning of the cables and to mitigate the cables from becoming disconnected from the associated sensors. However, it should be noted that other types of cable management could be utilized, either as part of the sensor holders or part of the helmet base or even as separate components to control the location of the cables and to mitigate disconnect of the same from the sensors.

Additional aspects of the plurality of sensor holders 21 includes the bottom 34 shown in FIG. 6. The bottom 34 may include a cutout 36 therethrough, which is sized and shaped similar to a sensor. This will place the sensor as close to the user's head as possible. It is preferred to have the distance between the inside of the helmet base and the bottom of the pocket to be small, but thick enough to not break.

According to at least some aspects and/or embodiments of the disclosure, the sensor holders 21 and corresponding pockets 15 of the helmet base 12 can be labeled or otherwise visually identified to indicate the location of each sensor holder/sensor on the helmet base 12. This will reduce the amount of time associated with placing the sensors at the appropriate location for use with the neuroimaging machine.

Similar to the helmet base 12, the sensor holders 21 may be D printed (or otherwise created using additive manufacturing), sculpted, carved, cut, molded, or formed out of any solid body(s) or body(s) that will harden into a solid body that is not magnetic. The material(s) used to make the sensor holder must be heat resistant up to at least the temperature that the OPM sensors that are being used heat up to.

FIG. 8 shows a neural imaging system 50 that includes a sensor system 40 according to aspects of the disclosure. The sensor system 40 includes the helmet system 10 as disclosed, including the helmet base 12 and sensor holders 21. This includes any of the variations of any of the embodiments discussed, disclosed, or shown. However, the figure shows additional aspects that can be included in at least some embodiments to further improve the time it takes to move sensors from one helmet to another, while also ensuring that the sensors and associated cables are oriented and connected to operate correctly.

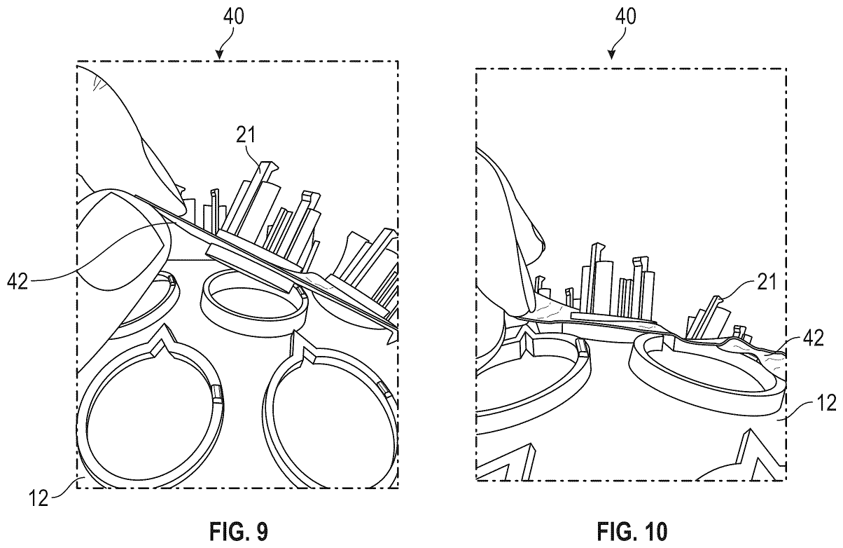

As shown in FIGS. 8-10, the sensor holders 21 can be connected to one another via a substrate 42. The substrate 42 connects each of the plurality of sensor holders 21 and thus, the associated sensors 52, to allow for quick and easy replacement when moving the sensors from one helmet to another.

If a substrate 42 is utilized, it is considered that the substrate comprise a flexible material, such as a fabric, that is attached to all of the sensor holders and any other cable management system (if others are used). A “fabric” can be defined as any thin, flexible material made from yarn, directly from fibers, polymeric film, foam, or any combination of these techniques. However, it should be appreciated that other flexible materials, including, but not limited to, polymers, plastics, rubbers, etc., could be utilized.

The substrate 42 is attached to the sensor holders 21 at any location. However, according to at least some embodiments, the substrate 42 can be attached to the holders at the length of the depth of the pocket 15 of the helmet base 12 above the base 22 of the sensor holder 21. This would position the substrate 42 at or near the rim 16 of the pockets 15 on the helmet base 12.

According to at least some embodiments of the disclosure, the fabric or other substrate can be attached with physical attachment point(s), chemically bonded, physically bonded, friction fit, or otherwise positioned relative to the sensor holders 21. According to at least some aspects of the disclosure, the substrate 42 is transparent or semi-transparent. This is so it is easier to see where the sensor holder 21 clips into the helmet base 12.

According to at least some embodiments of the disclosure, the substrate 42 is flexible enough to allow the sensor holders 21 that are attached to reach the pockets 15 that are in the helmet base 12.

According to at least some embodiments of the disclosure, the substrate 42 is heat resistant up to at least the temperature that the OPM or other neuroimaging machine being used heat up to.

Thus, the use of the substrate will create a type of sensor array for the helmet system, wherein the sensors in the sensor holders can be held and moved together. This will ensure that all of the sensors are moved, and also will aid in the placement of the sensors via the holders. The sensor array will also maintain cable management, which will improve efficiency of the system and mitigate disconnection of the cables from the sensors.

Utilizing any of the embodiments provided will keep the overall weight of the helmet light. In addition, it is considered that motion tracking markers may be placed on any part of the helmet if motion tracking is wanting to be used to track the helmet. Additional components to the helmet may be added for motion tracking markers. If motion tracking markers are used, they should be placed asymmetrically on the helmet.

In addition, it is noted that any of the components of any of the embodiments utilized should be non-magnetic. This will mitigate any noise or disruption of the system, which uses sensors that sense magnetic fields for operation.

Therefore, it should be appreciated that the present disclosure provides unique and advantageous features for helmets used with neuroimaging devices, such as MEGs and OPMs. Having the ability to detach the sensor holder with the cable management system instead of the sensor and cables from the helmet is a major improvement over the current systems that are offered and utilized. In addition, having the sensor holders attached to fabric/flexible material (i.e., the substrate) so that all the sensors can be taken off at once aids in the timeliness of switching helmets, and the people switching helmets do not have to deal with verifying the orientation of the sensor.

It should be appreciated that the system, methods, and/or apparatus as included herein will decrease the strain and stress on the cables that attach to the sensors because our cable management system secures the cable going straight out of the sensor. Keeping the components together will decrease the time to swap sensors from helmet to helmet. Still further, the cable management system mitigates the cables from twisting right next to the attachment point on the sensor and restricts the ability to pull the cable out of its socket. This should reduce the number of times cables break at that point and reduce the number of times the cable gets tugged out of its socket on the sensor. Still further, the system should decrease the amount of time it takes to check that the sensors are all functioning properly after we have changed the helmets.

As noted, the present disclosure, including any of the embodiments and any combination of any components of the embodiments, can be used with helmets for OPM machines. However, this could also be used with other body part scanning via an OPM. The helmet could be varied to cover any body part of a participant that could be scanned with an OPM or other imaging machine.

Additional alternatives, variations, and/or changes could be included. This could include, for example, the addition of channels in the helmet base for cables to lay in. This would keep the cables out of the way. It is preferred to have the whole helmet to have a low weight. Options to decrease the weight are to make the helmet base thinner, while still allowing structural stability, or add holes to any of the components while still allowing them to function as described in the disclosure. The aesthetics of this helmet design can be improved. Adding more pieces to the helmet may be able to hide the underlying components but it would also increase weight. Theoretically there is an optimal orientation that the sensors should be to get the widest coverage of brain activity. Sensors can also be put in different orientations to focus on one portion of the brain. To change the orientation, one would just need to change the orientation of the pockets on the helmet base.

Therefore, a helmet and associated systems for use with sensors of a neuroimaging machine have been shown and/or described. It should be appreciated that variations and/or changes to any of the components or embodiments that are obvious to those skilled in the art are to be considered a part of the present disclosure. In addition, any of the aspects of any of the embodiments disclosed could be combined in ways not explicitly shown and/or described to provide yet additional embodiments that are part of the disclosure. The disclosure is not to be limited to the embodiments disclosed herein.

1. A helmet system for use with a neural imaging machine, comprising:
a helmet base to cover at least a portion of a user's head; and
a plurality of sensor holders detachably connectable to the helmet base, each of the plurality of sensor holders configured to hold a sensor and including an orientation key for orienting the sensor holder relative to the helmet base.

2. The helmet system of claim 1, wherein the helmet base comprises a plurality of pockets, wherein the pockets are sized and oriented to receive a portion of one of the plurality of sensor holders.

3. The helmet system of claim 2, wherein the plurality of pockets each comprise an orientation key to aid in orientation of the sensor holders relative to the helmet base.

4. The helmet system of claim 2, wherein each of the plurality of pockets include a rim that extends away from the helmet base.

5. The helmet system of claim 1, wherein each of the plurality of sensor holders comprises one or more outwardly extending protrusions to hold the sensor therein.

6. The helmet system of claim 5, wherein one or more of the outwardly extending protrusions comprises a retaining lip for holding the sensor in place.

7. The helmet system of claim 1, wherein each of the plurality of sensor holders comprises a cable management system to hold a portion of a cable connected to a corresponding sensor positioned in a sensor holder.

8. The helmet system of claim 1, further comprising a substrate connected to the plurality of sensor holders to allow movement and selective attachment and detachment of the plurality of sensor holders as a substantially single unit.

9. The helmet system of claim 8, wherein the substrate comprises a flexible material.

10. The helmet system of claim 8, wherein the substrate comprises a fabric.

11. A sensor system for use with a helmet of a neural imaging device, comprising:
a plurality of sensor holders, each of the sensor holders comprising a base and one or more outwardly extending members, wherein each of the plurality of sensor holders are configured to hold a sensor such that the sensor is unable to substantially move relative to the sensor holder; and
a flexible substrate connecting each of the plurality of sensor holders to one another.

12. The sensor system of claim 11, wherein each base of the plurality of sensor holders comprises an asymmetrical shape.

13. The sensor system of claim 11, wherein one or more of the outwardly extending protrusions of the plurality of sensor holders comprises a retaining lip for holding the sensor in place.

14. The sensor system of claim 11, wherein each of the plurality of sensor holders comprises a cable management system to hold a portion of a cable connected to a corresponding sensor positioned in a sensor holder.

15. The sensor system of claim 11, wherein the flexible substrate comprises a fabric material.

16. The sensor system of claim 11, wherein the plurality of sensor holders and the flexible substrate are substantially non-ferromagnetic.

17. A neural imaging system, comprising:
a helmet base comprising a plurality of pockets;
a plurality of sensor holders detachably connectable to the helmet base at the plurality of pockets of the helmet base, each of the sensor holders comprising an orientation key to orient the sensor holder relative to a pocket of the helmet base; and
a plurality of sensors positioned in the plurality of sensors, wherein the plurality of sensors associated with a neural imaging machine.
a substrate connected to the plurality of sensor holders.

18. The neural imaging system of claim 17, further comprising a substrate connected to the plurality of sensor holders.

19. The neural imaging system of claim 17, wherein each of the plurality of pockets and a base of each of the plurality of sensor holders comprise a corresponding asymmetrical shape to aid in orienting a sensor held in each of the plurality of sensor holders.

20. The neural imaging system of claim 17, wherein each of the plurality of sensor holders comprises a cable management system to position a cable connected to a sensor of the plurality of sensors.
